(19) United States
(12) Patent Application Publication
(10) Pub. No.: US 2022/0151687 A1
(43) Pub. Date: May 19, 2022

(54) SYSTEM OF MEDICAL DEVICES AND METHOD FOR PERICARDIAL PUNCTURE

(71) Applicant: Baylis Medical Company Inc., Montreal (CA)

(72) Inventor: Gareth Davies, Toronto (CA)

(21) Appl. No.: 17/527,490

(22) Filed: Nov. 16, 2021

Related U.S. Application Data
(60) Provisional application No. 63/115,927, filed on Nov. 19, 2020.

(51) Int. Cl. A61B 18/14; A61B 18/12; A61B 90/30

(52) U.S. Cl. CPC A61B 18/1477; A61B 2018/00982; A61B 90/30; A61B 18/1206

(57) ABSTRACT

A medical device includes an elongate shaft extending between a proximal portion and a distal portion defining a distal end. The shaft includes a puncture device having a puncturing tip at the distal end, a distally facing camera in the distal portion, and a lighting system comprising distally facing light emitter in the distal portion.

### FIELD

This document relates to medical devices. More specifically, this document relates to medical devices that can be used in pericardial puncture, and related systems and methods.

### SUMMARY

The following summary is intended to introduce the reader to various aspects of the detailed description, but not to define or delimit any invention.

Medical devices are disclosed. According to some aspects, a medical device includes an elongate shaft extending between a proximal portion and a distal portion defining a distal end. The shaft includes a puncture device having a puncturing tip at the distal end, a distally facing camera in the distal portion, and a lighting system comprising distally facing light emitter in the distal portion.

In some examples, the shaft further includes an outer tube in which the puncture device is removably received. The outer tube can have a tube distal end, and the puncturing tip can be positioned proud of the tube distal end. The outer tube can include the light emitter and the camera.

In some examples, the puncture device includes a lumen extending therethrough from the proximal portion to the distal end, and the shaft further includes an inner shaft received in the lumen. The inner shaft can include the light emitter and the camera.

In some examples, the light emitter includes a light-guide lens, and the lighting system further comprises a bundle of optical fibers extending from the proximal portion toward the light-guide lens.

In some examples, the puncture device is a mechanical puncture device, and the puncturing tip includes a sharp tip. In some examples, the puncture device is a radiofrequency puncture device, and the puncturing tip includes an electrode.

In some examples, the medical device includes at least a first control for steering the distal portion.

Medical systems are disclosed. According to some aspects, a medical system includes a medical device and a viewing monitor. The medical device has an elongate shaft extending between a proximal portion and a distal portion defining a distal end. The shaft includes a puncture device having a puncturing tip at the distal end, a distally facing camera in the distal portion, and a lighting system comprising distally facing light emitter in the distal portion. The viewing monitor is for displaying images captured by the camera.

In some examples, the puncture device is a radiofrequency puncture device and the puncturing tip includes an electrode. The system can further include a radiofrequency generator connectable to the radiofrequency puncture device.

In some examples, the shaft includes an outer tube in which the puncture device is removably received. The system can further include a therapeutic device that is receivable in the outer tube.

In some examples, the outer tube includes a tube distal end, and the puncturing tip is positioned proud of the tube distal end.

In some examples, the outer tube includes the light emitter and the camera.

In some examples, the puncture device includes a lumen extending therethrough from the proximal portion to the distal end, and the shaft further includes an inner shaft received in the lumen. The inner shaft can include the light emitter and the camera.

In some examples, the light emitter includes a light-guide lens, and the lighting system further includes at least one optical fiber extending from the proximal portion toward the light-guide lens.

In some examples, the puncture device is a mechanical puncture device, and the puncturing tip includes a sharp tip.

In some examples, the medical device includes at least a first control for steering the distal portion.

Methods for pericardial puncture are also disclosed. According to some aspects, a method for pericardial puncture includes a. percutaneously advancing a medical device towards a pericardium; b. using a camera and a light emitter of the medical device to assess a position of a puncturing tip of the medical device; and c. puncturing the pericardium with the puncturing tip.

The method can further include advancing a therapeutic device through the medical device towards the pericardium.

Step b. can include capturing an image with the camera and displaying the image on a viewing monitor.

Step c. can include applying force to mechanically puncture the pericardium, or delivering radiofrequency energy to puncture the pericardium.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example medical system including a medical device and a control system;

FIG. 2 is a partial side view of the medical device of FIG. 1;

FIG. 3 is a cross-section taken along line 3-3 in FIG. 2;

FIG. 4 is a cross-section similar to that of FIG. 3, showing a puncture device removed from an outer tube of the medical device and a therapeutic device received in the outer tube;

FIG. 5 is a partial side view of another example medical device;

FIG. 6 is a cross-section taken along line 6-6 in FIG. 5;

FIG. 7 is an end view of the medical device of FIG. 5; and

FIG. 8 is a perspective view of another example medical system.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are medical devices (and related systems and methods) that can be used for pericardial puncture. The medical devices are configured both to allow for imaging of the surroundings of the device (e.g. of the pericardium and/or of the puncturing tip of the device), and to puncture the pericardium (e.g. by mechanical or radiofrequency (RF) puncture). By combining imaging ability with puncture ability, the the target site for puncture may be more readily identified by the user, and patient safety may be enhanced.

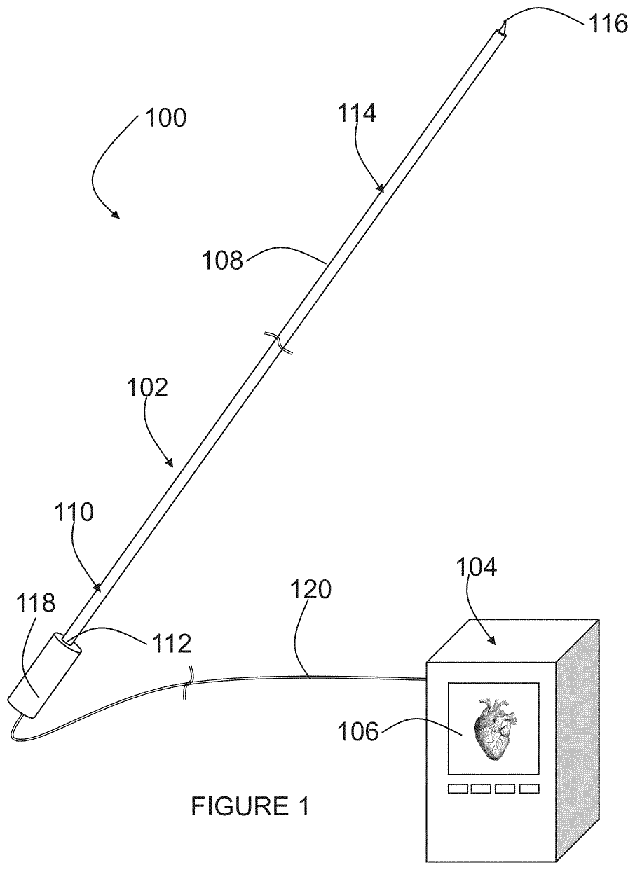

Referring now to FIG. 1, a medical system 100 is shown. The medical system 100 generally includes a medical device 102, and a control system 104.

In the example shown, the control system 104 includes a processor (not shown) that runs image processing software, and a viewing monitor 106. The viewing monitor 106 can be of any configuration that can display images (e.g. still images or video images) captured by the medical device 102, and is not described in detail herein.

In the example shown, the medical device 102 includes an elongate shaft 108 that extends between a proximal portion 110 defining a proximal end 112, and a distal portion 114 defining a distal end 116. A handle 118 is mounted to the proximal portion 110. A cable 120 extends from the handle 118 to the control system 104, for connecting the components of the medical device 102 to the control system 104.

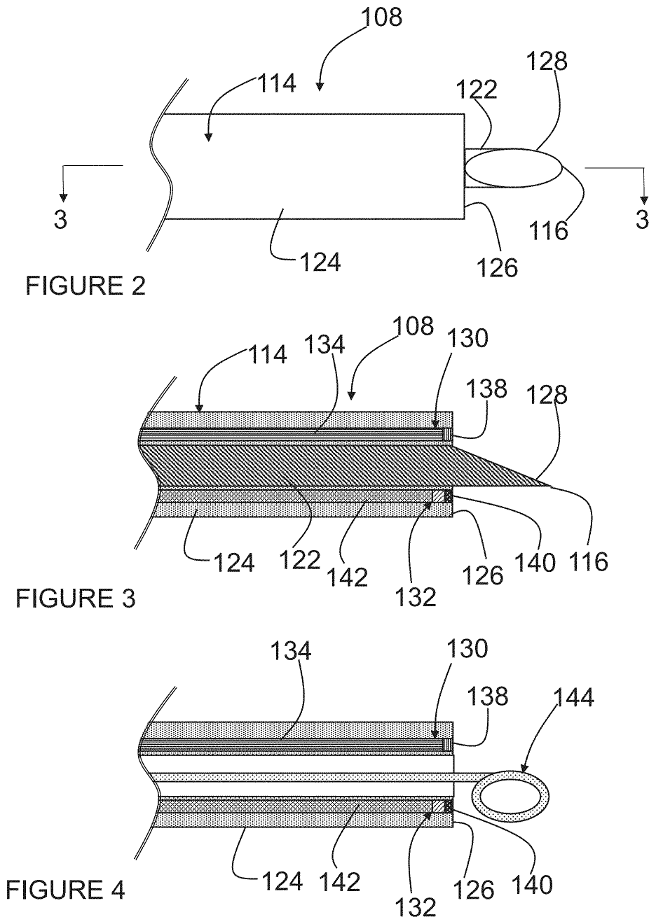

Referring now to FIGS. 2 and 3, in the example shown, the shaft 108 includes a puncture device 122, and an outer tube 124. The outer tube 124 may be, for example, a polymeric tube that includes one or more polymeric layers (e.g. layers of high-density polyethylene or low-density polyethylene) and/or one or more braided layers. The outer tube 124 may be relatively durable, and may be generally rigid or flexible. The outer tube 124 includes a tube distal end 126.

The puncture device 122 is for puncturing tissue, particularly the pericardium, and in the example shown is removably received in the outer tube 124. The puncture device 122 may be a metallic hypotube, and includes a puncturing tip 128 that is positioned at the distal end 116 of the shaft 108, proud of the tube distal end 126. In the example shown, the puncture device 122 is a mechanical puncture device, and the puncturing tip 128 is a sharp tip.

Referring still to FIG. 3, the shaft 108 further includes a lighting system 130 and a camera 132, which are positioned in the distal portion 114 of the shaft 108 and are embedded in the outer tube 124.

The lighting system 130 is for illuminating the distal portion 114 and/or the surroundings thereof (e.g. for illuminating the puncturing tip 128 and/or the pericardium), and in the example shown includes a bundle 134 of optical fibers that are connected to a light source (not shown) and that extend through the outer tube 124 from the proximal portion 110 to the distal portion 114, and a light emitter 138 in the form of a light-guide lens that is distally facing, for guiding light from the bundle 134 of optical fibers to the exterior of the device 102. The light source may be housed in the control system 104, and may be, for example, a bright xenon lamp that emits white light, (such as quartz halogen).

The camera 132 is for capturing images (e.g. still images or video images) of the distal portion 114 and/or the surroundings of the distal portion 114, and in the example shown includes an objective lens 140 that is distally facing. The objective lens 140 can optionally be movable, e.g. to allow for parfocal zoom and image enlargement. The camera 132 can be, for example a charge coupled camera that converts optical information into electrical signals. The camera 132 can be a three-chip camera, with one channel for each of the primary colors. In the example shown, the camera 132 is connected to the control system 104 via signal wires 142, which extend through the outer tube 124 from the proximal portion 110 to the distal portion 114.

In alternative examples, the light-emitter 138 and/or camera 132 can face in other directions, such as radially outwardly, or in an oblique direction.

Referring to FIG. 4, as mentioned above, in the example shown the puncture 122 (not shown in FIG. 4) device is removably received in the outer tube 124. The puncture device 122 can be removed from the outer tube 124 after puncture is complete, and a therapeutic device 144 can optionally then be advanced through the outer tube 124, e.g. to allow for a therapy to be applied.

Referring back to FIG. 1, the medical device 102 can optionally be steerable. For example, the handle 118 can be used to steer the distal end 116 (e.g. to deflect the distal end 116). This can be achieved, for example, by turning the handle 118 to actuate control wires (not shown) connected to the distal end 116. The handle 118 can further optionally include buttons (or another mechanism, not shown) for controlling the camera 132 and/or the lighting system 130 (e.g. for freezing images, changing the wavelength of emitted light, or altering the focus of the camera 132), or for changing the stiffness of the outer tube 124.

In general, in use, the medical device 102 can be percutaneously advanced towards a target site, such as the pericardium. The medical device 102 can be advanced via a relatively small incision, as the medical device 102 allows for imaging of the surroundings of the distal portion 114 to identify the target site, and thus direct viewing of the target site may not be necessary. During advancement, the position of the puncturing tip 128 can be assessed by using the light emitter 138 to illuminate the distal portion 114 and/or the surroundings of the distal portion 114, and by using the camera 132 to obtain images of the distal portion 114 and/or the surroundings of the distal portion 114. That is, images can be captured by the camera 132 and can be displayed on the viewing monitor 106, to allow the user to assess the position of the puncturing tip 128. When it is determined from the images that the puncturing tip 128 is in the desired position, e.g. adjacent the pericardium, the puncturing tip 128 can be used to puncture the pericardium (e.g. by applying force). When puncture is complete, the puncture device 122 can optionally be withdrawn from the outer tube 124, and a therapeutic device can be advanced through the outer tube 124, to perform a treatment from within the pericardial space.

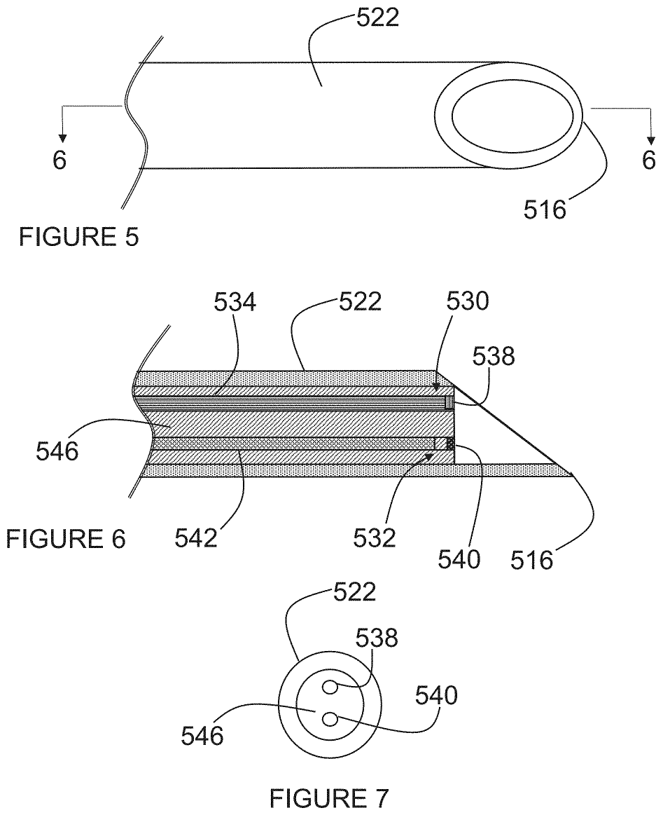

Referring now to FIGS. 5 to 7, another example of a medical device is shown. In FIGS. 5 to 7, features that are like those of FIGS. 1 to 4 will be identified with like reference numerals, incremented by 400.

In the medical device of FIGS. 5 to 7, the puncture device 522 includes a lumen (not shown) that extends from the proximal end (not shown) to the distal end 516, and an inner shaft 546 (which may be made of similar materials to the outer tube 124 as described above) is received in the lumen. The lighting system 530 (including the bundle 534 of optical fibers and the light emitter 538) the camera 532 (including the objective lens 540) and the signal wires 542 are embedded in the inner shaft 546.

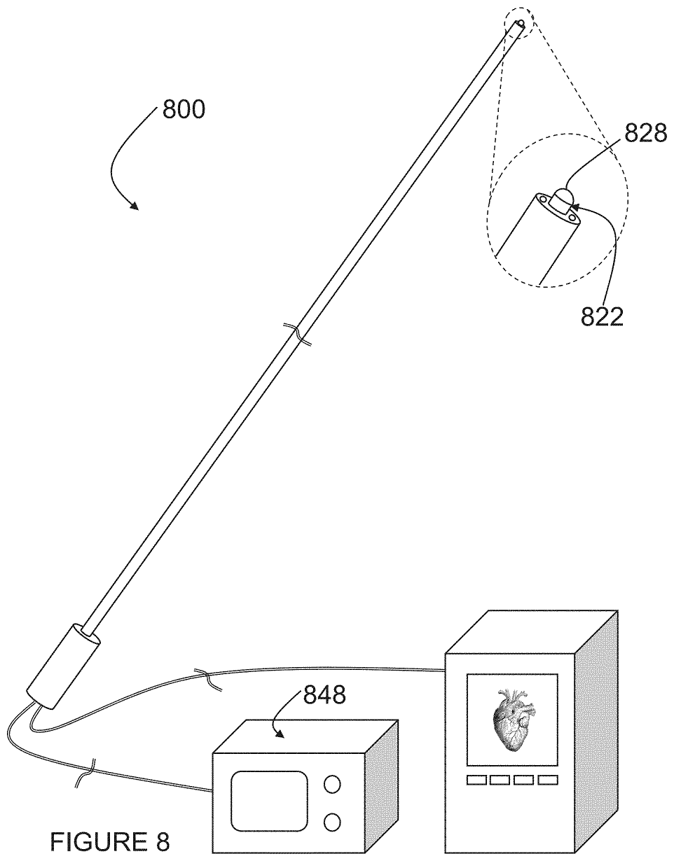

Referring now to FIG. 8, another example of a medical system is shown. In FIG. 8, features that are like those of FIGS. 1 to 4 will be identified with like reference numerals, incremented by 700.

In the system 800, the puncture device 822 is a radio frequency puncture device, and the puncturing tip 828 includes an electrode. The system further includes an RF generator 848, which is electrically connected to the electrode (e.g. via a cable and an electrical connector embedded in the outer tube), for delivering RF energy to the electrode.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A medical device, comprising:
an elongate shaft extending between a proximal portion and a distal portion defining a distal end;
the shaft comprising a puncture device having a puncturing tip at the distal end, a distally facing camera in the distal portion, and a lighting system comprising distally facing light emitter in the distal portion.

2. The medical device of claim 1, wherein the shaft further comprises an outer tube in which the puncture device is removably received.

3. The medical device of claim 2, wherein the outer tube comprises a tube distal end, and the puncturing tip is positioned proud of the tube distal end.

4. The medical device of claim 2, wherein the outer tube comprises the light emitter and the camera.

5. The medical device of claim 1, wherein the puncture device comprises a lumen extending therethrough from the proximal portion to the distal end, and the shaft further comprises an inner shaft received in the lumen, wherein the inner shaft comprises the light emitter and the camera.

6. The medical device of claim 1, wherein the light emitter comprises a light-guide lens, and the lighting system further comprises a bundle of optical fibers extending from the proximal portion toward the light-guide lens.

7. The medical device of claim 1, wherein the puncture device is a mechanical puncture device, and the puncturing tip comprises a sharp tip.

8. The medical device of claim 1, wherein the puncture device is a radiofrequency puncture device, and the puncturing tip comprises an electrode.

9. The medical device of claim 1, further comprising at least a first control for steering the distal portion.

10. A medical system comprising:
a medical device comprising an elongate shaft extending between a proximal portion and a distal portion defining a distal end, the shaft comprising a puncture device having a puncturing tip at the distal end, a distally facing camera in the distal portion, and a lighting system comprising distally-facing light emitter in the distal portion; and a viewing monitor for displaying images captured by the camera.

11. The medical system of claim 10, wherein
the puncture device is a radiofrequency puncture device and the puncturing tip comprises an electrode; and
the system further comprises a radiofrequency generator connectable to the radiofrequency puncture device.

12. The medical system of claim 10, wherein the shaft further comprises an outer tube in which the puncture device is removably received, and said outer tube comprises the light emitter and the camera.

13. The medical system of claim 12, further comprising a therapeutic device receivable in the outer tube.

14. The medical system of claim 12, wherein the outer tube comprises a tube distal end, and the puncturing tip is positioned proud of the tube distal end.

15. The medical system of claim 10, wherein the puncture device comprises a lumen extending therethrough from the proximal portion to the distal end, and the shaft further comprises an inner shaft received in the lumen, wherein the inner shaft comprises the light emitter and the camera.

16. The medical system of claim 10, wherein the light emitter comprises a light-guide lens, and the lighting system further comprises at least one optical fiber extending from the proximal portion toward the light-guide lens.

17. The medical system of claim 10, wherein the puncture device is a mechanical puncture device, and the puncturing tip comprises a sharp tip.

18. A method for pericardial puncture, comprising:
a. percutaneously advancing a medical device towards a pericardium
b. using a camera and a light emitter of the medical device to assess a position of a puncturing tip of the medical device;
c. puncturing the pericardium with the puncturing tip; and
d. advancing a therapeutic device through the medical device towards the pericardium.

19. The method of claim 18, wherein step c. comprises applying force to mechanically puncture the pericardium.

20. The method of claim 18, wherein step c. comprises delivering radiofrequency energy to puncture the pericardium.
